(19) United States
(12) Patent Application Publication
(10) Pub. No.: US 2021/0082305 A1
(43) Pub. Date: Mar. 18, 2021

(54) DATA-DRIVEN SLEEP COACHING SYSTEM

(71) Applicant: RESMED SENSOR TECHNOLOGIES LIMITED, Clonskeagh (IE)

(72) Inventors: David DICKINSON, Sudbury, MA (US); Jason DONAHUE, Boston, MA (US); Stephan FABREGAS, Boston, MA (US); Benjamin RUBIN, Boston, MA (US); John SHAMBROOM, Framingham, MA (US); Eric SHASHOUA, Norwood, MA (US)

(73) Assignee: RESMED SENSOR TECHNOLOGIES LIMITED, Clonskeagh (IE)

(21) Appl. No.: 16/948,026

(22) Filed: Aug. 27, 2020

Related U.S. Application Data
(63) Continuation of application No. 13/974,358, filed on Aug. 23, 2013, now abandoned.
Application No. 13/974,358 is a continuation of application No. 12/387,730, filed on May 6, 2009, now abandoned.
(60) Provisional application No. 61/196,960, filed on Oct. 22, 2008.

(51) Int. Cl. G09B 19/00; A61B 5/00; A61B 5/0476; G16H 50/20

(52) U.S. Cl. CPC G09B 19/00; A61B 5/6814; A61B 5/4815; G16H 50/20; A61B 5/4812; A61B 5/0006; A61B 5/0476

(57) ABSTRACT

System and method for a user to monitor and/or modify his or her sleep. In one embodiment, the sleep coaching system comprises a sensor for sensing a physiological signal of a sleeping user such as an EEG, computer memory databases for storing user and sleep-related data and advice, and a processor that generates a set of advice to improve user sleep satisfaction based on the user and sleep-related data. The advice to improve user sleep satisfaction, which may be communicated to the user, may comprise a sleep coaching plan, which may include one or more sleep coaching workshops that the user may undertake.

### CROSS-REFERENCE TO RELATED APPLICATIONS

This application claims the benefit of U.S. Provisional Application No. 61/196,960 filed Oct. 22, 2008, which is hereby incorporated by reference herein in its entirety.

### BACKGROUND

It is well understood that sleep plays an important role in learning and memory. Despite this, most people tend to not get enough sleep, and when they do sleep, the sleep is often reported to be of poor quality. This lack of high quality sleep may lead to decreased quality of life and decreased performance in critical tasks. Many individuals sleep poorly due to a lack of understanding of the factors that affect their sleep quality, such as sleep hygiene, sleep stages, etc. Current methods and systems to help people get a better night's sleep tend to provide broad recommendations and suggestions that are not personalized for a particular user and therefore not as useful as personalized advice. Even methods that can provide personalized sleep instruction and advice, such as visiting a sleep coach or participating in a sleep study, can be laborious and time-consuming. Thus, there remains a need for systems and methods that improve a person's sleep satisfaction.

### SUMMARY OF THE INVENTION

The systems and methods described herein include more particularly, an easy-to-use, automated sleep coaching system that can provide a personalized sleep coaching plan for a particular user. The systems and methods described herein provide data-driven sleep coaching to a user. In one embodiment, the system comprises a headband-mounted first sensor that senses a first physiological signal associated with a sleeping user, such as an electroencephalogram (EEG). The first sensor may be dry, require no preparation, and be easy to apply with a lightweight headband. The first sensor may transmit the sensed first physiological signal to a first processor such as a base station. The base station may process the received first signal or not, for example by using a Fast Fourier Transform (FFT) to convert the received signal into its constituent frequency bands, but in either case, it transmits the resulting second data set to a second processor such as a host computer. In addition to receiving the second data set from the base station, the host computer may receive one or more indications of user behavior or user characteristics, such as user bedtime, user risetime, or other user sleeping or eating habits. This may be done in the form of a computer-based questionnaire. The host computer may then generate advice for improving user sleep satisfaction such as a sleep coaching plan based at least in part on at least one of the second sleep data set, the one or more indications of user behavior or characteristics, and a database containing sleep-related data and advice. This sleep coaching plan may comprise one or more sleep coaching workshops, which the user may undertake. In one embodiment, the system may also comprise a third processor located remotely from the user, such as a remote server. The third processor mentioned here could be an expert human operator or an automated expert system. The host computer may transmit a third data set based on the second data set to the remote server.

In certain embodiments the second processor may be the remote server. In this case, the remote server may be configured to receive the one or more indications of user behavior or characteristics instead of the host computer, for example through a network or internet interface such as a website. The host computer may act as a way station, forwarding the second data set received from the base station to the remote server through a network or internet interface. The generation of the advice for improving user sleep satisfaction may occur at the remote server instead of at the host computer.

In one embodiment, the first signal, second data set, and third data set may be transmitted via any suitable wireless or wired transmission method, such as radio frequency (RF), infra-red (IR), Bluetooth, WiFi, USB, Ethernet, or other similar interfaces. In one embodiment, the second data set may be transferred via a storage device such as a portable USB flash drive, a Secure Digital (SD) card, or other similar storage devices.

In certain embodiments, the first processor and the second processor may be located in the same housing. For example, a personal computer may act as both the base station, or first processor, and the host computer, or second processor. In another embodiment, the remote server may act as the first and second processor, and be located at a central location geographically remote from the user.

In certain embodiments, the first processor may display the first signal to the user on a display such as a television, computer monitor, or other similar display. The display may be in the same housing as the first processor. The first signal may be displayed in a form such as a hypnogram. In one embodiment, the display may also display data such as the current time. Similarly, the generated advice for improving user sleep satisfaction may be displayed to the user on a display such as a television, computer monitor, or other similar display. In one embodiment, the generated sleep-related recommendation may be displayed to the user on a website accessible on a network, such as a local area network (LAN), wide area network (WAN), or the Internet. In another embodiment, the generated sleep-related recommendation may be displayed to the user by sending an email accessible on a network, such as a local area network (LAN), wide area network (WAN), or the Internet.

The first or second processors may have a user interface. The user interface may be a remote control, a keyboard, a touchscreen, or other similar interface.

The user behavior or characteristics may comprise at least one of age, gender, sleeper type/subtype, sleep hygiene, and sleep diary.

One or more sleep coaching workshops may comprise personalized advice generated based at least on the first set of sleep data, such as a recommended bed time, or a limit on caffeine consumption. In certain embodiments, a sleep coaching workshop may relate to a specific user sleep-related issue identified from gathered user sleep or behavior data. User sleep-related issues may comprise issues such as difficulty falling asleep after consumption of caffeine or difficulty staying asleep after consumption of alcohol. In certain embodiments, a sleep coaching workshop may comprise a user questionnaire related to a specific user sleep-related issue, one or more pieces of sleep-related advice, and a summary of results generated based on user sleep performance during the workshop. Sleep-related advice may comprise advice such as abstaining from caffeine or alcohol after noon, or refraining from exercising several hours before bedtime. The summary of results may comprise sleep parameter changes resulting from adoption of a piece of sleep-related advice, such as improved user sleep satisfaction resulting from abstention from caffeine. Sleep satisfaction could be based on objective changes in sleep data or be based on a user's subjective assessment of their own sleep.

In one aspect, the invention provides a kit for an interactive sleep coaching program. The kit comprises a sleep sensor of the type that measures a physiological signal and generates and displays sleep data that characterizes a user's sleep. The kit further comprises a sleep coaching program for collecting information about the user's sleeping conditions and for selecting as a function of an algorithm that considers the collected information, a targeted set of advice stored within a data base of stored advice, for improving the sleep satisfaction of the user, whereby the user may collect advice from the sleep coaching program and employ the sleep sensor to determine interactively whether the advice and sleep coaching program are improving their sleep satisfaction.

In certain embodiments, the sleep coaching program includes means for collecting user data respective of at least one of demographic data and lifestyle data. Optionally, the sleep coaching program includes means of collecting data representative of the user sleep data. In certain embodiments, the sleep coaching program includes means for collecting data representative of user goals for improving sleep satisfaction and employs these goals when selecting advice.

In certain embodiments, the sleep coaching program collects data from the sensor representative of a baseline measure of user sleep quality. Optionally, the sleep coaching program generates an assessment of changes in sleep quality as a function of a previous measure of sleep data and subsequent measures of user sleep data. In certain embodiments, the sleep coaching program generates periodic assessments as a function of milestones within the sleep coaching program, a measured baseline of user sleep quality, and/or a normalized baseline representative of a normative sleep quality measure of a predetermined population. Optionally, the sleep coaching program allows the user to enter sleep data for providing feedback to the sleep coaching program to select subsequent advice from the data base and/or collects diary data from the user representative of events in the user's life over a selected time period that affect the user's sleeping conditions. In all of the above embodiments, the kit may further include means for communicating with a live sleep coach and exchanging sleep data of the user and receiving expert advice from the live sleep coach.

In another aspect, the invention provides an interactive sleep coaching system. The interactive sleep coaching system comprises a sensor of the type that can be worn by a user to measure a physiological signal to collect user sleep data and a table-top processor unit for communicating with the sensor and recording the sleep data collected by the sensor over a defined period of time. The table-top processor unit includes a baseline processor for generating a baseline representative of sleep quality of the user. The interactive sleep coaching system further comprises a user data input device for collecting diary data indicative of events in the user's life and the timing of those events, a processor for correlating, at least as a function of time, the recorded sleep data with the collected diary data to generate a first set of advice for improving the sleep satisfaction based at least in part on the sleep data associated with the defined period of time, and a progression processor for collecting sleep data over a second later period of time and providing to the user a second set of sleep advice for improving the sleep satisfaction based at least in part on the sleep data associated with the second later period of time and the first set of advice.

In certain embodiments, the progression processor includes means for adjusting the baseline as a function of sleep data collected over the second alter period of time, to revise the baseline to reflect changes in sleep over time.

In yet another aspect, the invention provides a method for providing an interactive sleep coaching program to a user. This method includes receiving sleep data associated with a first day sleep data associated with a second day and being indicative of quality of sleep, wherein the sleep data is determined by sensing and processing a physiological signal of the user while the user is sleeping. This method also includes receiving diary data indicative of user lifestyle events, the diary data including data received from the user describing lifestyle events during the first day and data received from the user describing lifestyle events during the second day. This method further includes mapping the sleep data associated with the first day to the diary data associated with the first day, providing to the user a first set of advice for improving user sleep satisfaction based at least in part on the sleep data associated with the first day, mapping the sleep data associated with the second day to the diary data associated with the second day, and providing to the user a second set of advice for improving user sleep satisfaction based at least in part on the sleep data associated with the second day and the first set of advice.

In all of the above aspects and embodiments, the physiological signal may be an electroencephalogram or electroencephalogram signal. The physiological signal may also be movement, respiration, heart rate, heart rate variability, peripheral arterial tone, galvanic skirt response, temperature, etc. In all of the above aspects and embodiments, the first set of advice for improving user sleep satisfaction may include a sleep coaching plan. The sleep coaching plan includes at least one sleep coaching workshop directed to at least one sleep-related issue generated based at least in part on at least one of the first physiological signal and the indication of user behaviors or user characteristics. The at least one sleep coaching workshop includes a questionnaire, at least one piece of advice to improve user sleep quality, and a summary of results based at least in part on the first physiological signal received during the workshop.

### BRIEF DESCRIPTION OF THE DRAWINGS

The invention may be better understood from the following illustrative description, taken in conjunction with the accompanying drawings in which:

FIG. 1A shows an exemplary data driven sleep coaching system, according to an illustrative embodiment of the invention;

FIG. 1B shows an alternative data driven sleep coaching system, according to an illustrative embodiment of the invention;

FIGS. 2 and 3 are block diagrams of an exemplary data driven sleep coaching system, according to an illustrative embodiment of the invention;

FIG. 4 shows an exemplary hypnogram, according to an illustrative embodiment of the invention;

FIG. 5 is a flow chart of steps involved in an exemplary sleep coaching program, according to an illustrative embodiment of the invention; and

FIG. 6 is a flow chart of steps involved in an exemplary method for generating sleep-related advice to improve user sleep quality, according to an illustrative embodiment of the invention.

### DETAILED DESCRIPTION OF ILLUSTRATIVE EMBODIMENTS

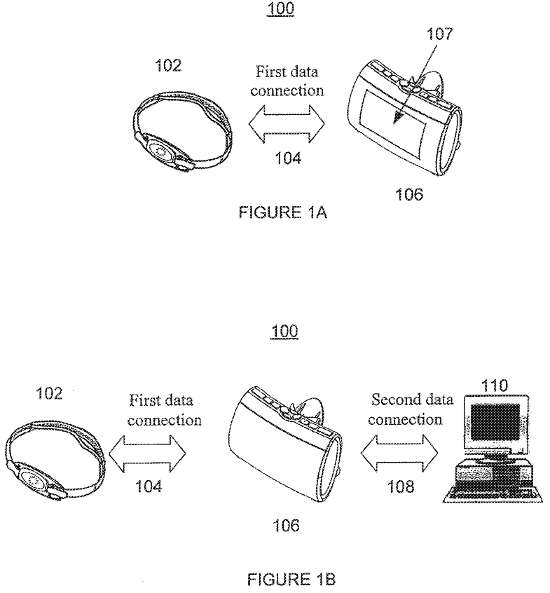

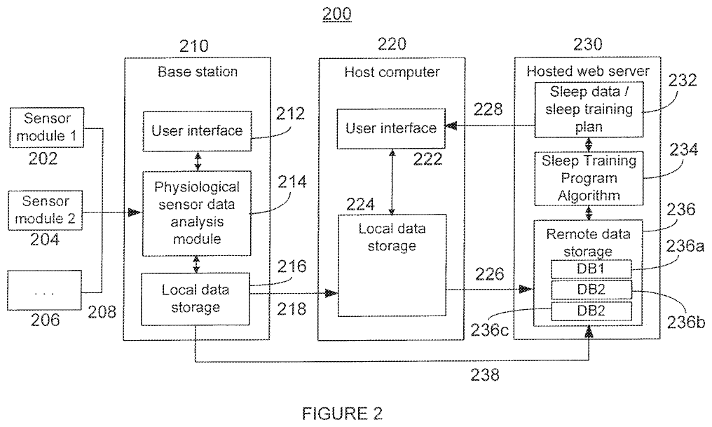

FIG. 1A depicts an exemplary data-driven sleep coaching system 100 comprising three modules, according to an illustrative embodiment. A first sensor 102 may be linked to a base station 106 via a first data connection 104. In an alternative data-driven sleep coaching system shown in FIG. 1B, the base station 106 may optionally be linked to a host computer 110 via a second data connection 108. In one embodiment, the second data connection 108 may involve a portable memory device such as a Secure Digital (SD) media card or a USB flash drive to transfer data from the base station 106 to the host computer 110.

The sensor 102 may have electrodes or other sensors for sensing one or more user physiological signals. In one embodiment, the sensor 102 has at least one electrode for sensing an electroencephalogram (EEG). In certain embodiments, sensor 102 may have one or more sensors for sensing one or more of electroencephalograms, electrooculograms, electromyograms, pulse rate, respiration rate, body movement, or any other user physiological signal. In certain embodiments, sensor 102 may be in the form of a flexible or rigid band that may be fastened around some portion of the user, such as the wrist, ankle, waist, or head. A sensor 102 that is worn by the user may include soft flexible head bands, such as the depicted sensor 102 of FIGS. 1A and 1B. In one particular embodiment, the sensor 102 includes one or more soft electrically conductive biosensors that may make contact with the patient's skin. The user may tighten the head band so that the soft conductive sensors are put in contact with the user's skin, with the contact being sufficient to all the electrical conductive sensors to record electro-physiological signals, or any suitable signal of the user.

Accordingly, FIGS. 1A and 1B depict an interactive sleep coaching system that has a head band sensor 102 that the user wears to allow the system 100 to measure a physiological signal, such as an EEG signal. The measured physiological signals may be analyzed or otherwise processed to collect user sleep data. For example, in the embodiment having an EEG head band sensor, the sensor 102 measures an EEG signal. Measured EEG signal is passed to the processor 106, which in this embodiment is a table top bedside unit. As depicted by the data exchange arrow 104, the sensor 102 and the depicted table top processor 106 exchange data. The data exchange is sufficient to at least transmit data representative of the measured physiological signal from the head band sensor 102 to the depicted table top bedside processor unit 106. In other embodiments, sensor 102 may include one or more non-contact sensors that may be able to sense user physiological signals. Exemplary sensor modules are further described in U.S. application No. 11/586,196 filed Oct. 24, 2006, Ser. No. 11/499,407 filed Aug. 4, 2006, and Ser. No. 11/069,934 filed Feb. 28, 2005, the entireties of which are hereby incorporated by reference herein.

In certain embodiments, the base station 106 may include a display 107. Display 107 may be used to display information to the user, or may be used by the user in conjunction with a user interface (not shown) to provide information to the base station 106. In certain embodiments, display 107 may be a touchscreen display, and the user interface may be integrated into the display 107.

The base station 106 and the host computer 110 may optionally reside in the same housing (not shown). For example, a personal computer may act as both the base station 106 and the host computer 110.

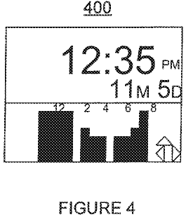

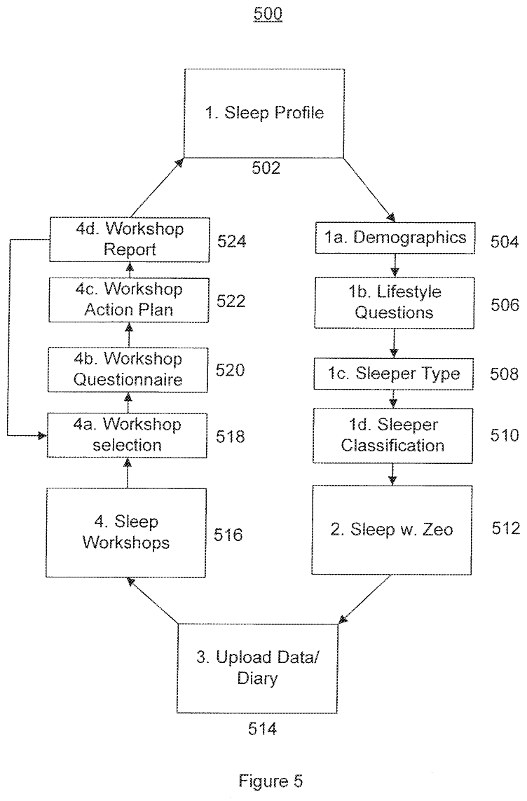

In such an embodiment, the processor unit 106 includes a conventional data memory for storing the recorded physiological signal. The process unit 106 also includes a programmed microprocessor or other data processing device for processing the raw physiological signal to generate sleep data. To this end, the processor unit 106 processes the measured physiological signal to generate a set of metrics that quantitatively measure physical characteristics of the user's sleep event, where the sleep event is a defined sleeping event, such as a night of sleep or an daily nap. For example, the processor unit 106 may process the physiological signal to determine a time at which the user started to sleep and a final time representation of when the user stopped sleeping for a defined sleep event, such as the sleep events that occurred during the night hours or during some other defined periods of time. In one particular embodiment, the process unit 106 includes a baseline processor for generating a baseline measure representative of sleep quality of the user. The baseline measure may be determined as described with reference to FIGS. 4 and 5 and optimally displayed to the user. In this way, the system 100 gives the user feedback representative of the quality of their sleep.

In this optional embodiment where the system 100 is incorporated into a personal computer, the processor unit 106 has a user interface that allows a user to answer survey questions about their current physical conditions, such as their age, gender, and general health. The user can enter additional information, such as information about their stress levels, or the hours that they typically work during the day or week. The user can enter information about their sleep habits, note specific habits, describe their sleeping environment, such as whether they have a sleeping partner, or room darkening shades, or note events surrounding their sleep. Further optionally, they can also keep a sleep diary. The sleep diary would collect information about a users consumption before bed on a particular day, their anxiety level on that same day, and whether they remember being dist///urbed by a bed partner that night. The survey and optional sleep diary information provide information about physical and psychological characteristics of the user.

Optionally, the processor 106 may have a database of stored information, typically advice, for improving the user's sleep satisfaction. The database may be any suitable database and the processor 106 will have a database management system that allows data stored within the database to be selected and presented to the user.

The database can be accessed by a sleep coaching computer program that analyzes the information about that respective user's sleeping conditions and selects from the database advice that is tailored to the user's particular characteristics. To this end, the processor 106 selects the advice by operation of an algorithmic process that considers the survey information about the user. In this way, the user is presented with targeted advice selected by the process to address their situation. As an example, a user who sleeps with a bed partner who disturbs their sleep would be given advice to cope with this difficulty, where a user who sleeps in bed alone would be given different advice. Optionally, as will be discussed in more detail below, the system can select advice based on the sleep data and the user characteristics determined from the user survey.

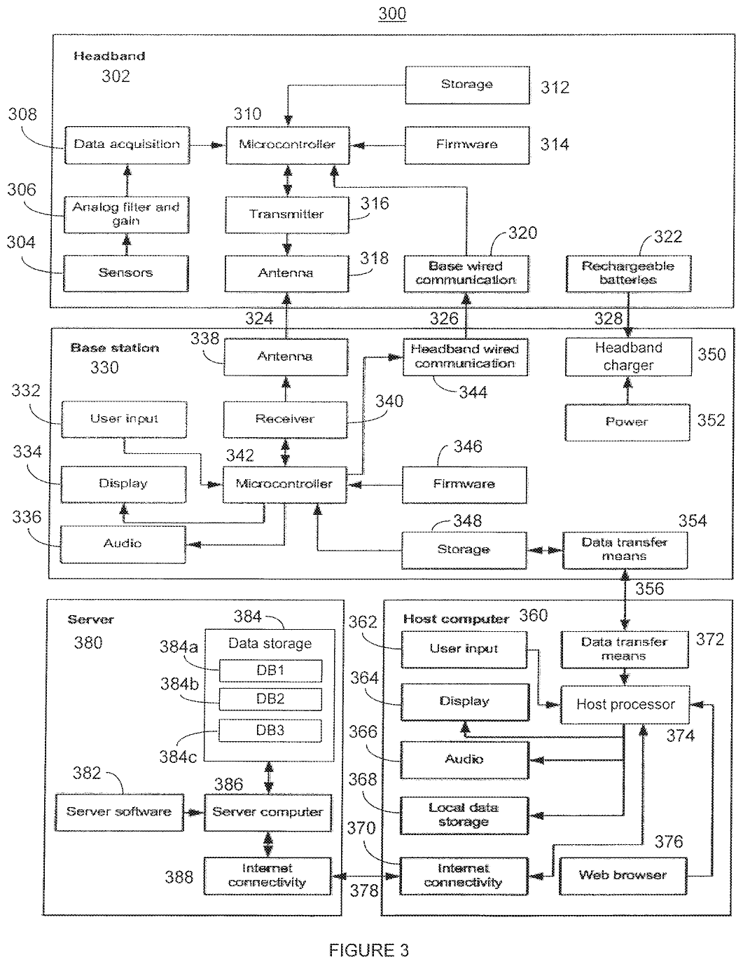

FIG. 2 is a functional block diagram of an exemplary data-driven sleep coaching system with a remote server component. One or more sensor modules 202, 204, and/or 206 may be linked to a base station 210 via a first data connection 208, which may be any type of wired or wireless connection known to those skilled in the art, such as radio frequency (RF), Bluetooth, WiFi, infra-red, wired USB, Ethernet, serial, or other similar interfaces. The sensor modules 202, 204, and/or 206 may be configured to sense one or more user physiological signals, which may then be transmitted to base station 210. The sensor modules 202, 204, and/or 206 may be further configured to condition the sensed physiological signals before transmission to base station 210.

Base station 210 may have a user interface 212, a sensor data analysis module 214, and local data storage 216. User interface 212 may include user input devices such as a keyboard, a touchscreen, an array of buttons, or a radio frequency or infra-red link to a remote control input device. User interface 212 may also include devices for communicating data to the user visually and/or audibly, such as a display screen or a speaker. Sensor data analysis module 214 may be configured to receive data from one or more sensor modules 202, 204, and/or 206 from data connection 208 and/or the user interface 212. Sensor data analysis module 214 may generate a first set of sleep data indicative of quality of sleep from the sensor data received via data connection 208 by converting sensor data into data that may represent metrics of sleep quality and quantity and may be more compact in memory footprint. Sleep data may be collected from monitoring the user. The sleep data typically includes a set of metrics that quantitatively measure physical characteristics of the user's sleep event, where the sleep event is a defined sleeping event, such as a night of sleep or an daily nap. The metrics that can be used by the coaching systems and methods described herein are illustrated and described with, among other places, reference to FIGS. 4 and 5.

In one embodiment, the sensor data may be raw EEGs. The sensor data analysis module may use a digital processing mechanism such as Fast Fourier Transform (FFT) to convert the raw EEG data into its constituent frequency bands. Then a neural net approach may be used to convert the frequency band information into stages of sleep on an epoch by epoch basis, where each epoch may be a slice of time from 30 seconds to 2 minutes long. In certain embodiments, the sensor data analysis module may also generate and store a first set of sleep parameters representing user sleep quality, such as total time spent sleeping, the breakdown of time spent in various stages of sleep, and the computation of a single sleep score to represent the quality of sleep. The sleep stage at each epoch may be stored in the form of a hypnogram. The user interface 212 may be used to present this information to the user for instant feedback.

The received and generated data may be stored in local data storage 216. Local data storage 216 may be physical memory embedded within base station 210 which may include, but is not limited to, one or more hard drives or random access memory (RAM), or a portable memory device which may include, but is not limited to, SD cards, mini SD cards, micro SD cards, XD cards, CompactFlash memory, Memory Stick, Memory Stick Duo, or any other such types of miniaturized portable memory devices. This stored data may then be transmitted to host computer 220 via second data connection 208. In one embodiment, the second data connection 208 may involve a wireless interface between the base station and the computer. The wireless interface may involve a standard radio frequency link, where a radio frequency dongle may be plugged into the host computer via a standard input/output port such as a USB port. A proprietary protocol may be used to transmit the sleep data from the base station 210 to the host computer 220. Other wireless protocols may be used, such as Bluetooth® wireless technology, WiFi, infra-red, or other standard wireless data transport mechanisms.

In certain embodiments, the second data connection 208 may involve a wired interface between the base station 210 and the host computer 220. The wired interface may utilize a standard port on the computer, such as the USB port, the firewire port, the parallel port, or other types of data ports for data uploading.

In certain embodiments, a portable memory device may be utilized to store the data within the base station 210. For example, the portable memory device may be plugged into a receptacle in the base station 210 for data capture over several nights. This portable memory device may then be extracted and plugged into a card reader that is connected to the host computer 220 for data uploading. Suitable portable memory devices may include, but are not limited to, SD cards, mini SD cards, micro SD cards, XD cards, CompactFlash memory, Memory Stick, Memory Stick Duo, or any other such types of miniaturized portable memory devices. In yet another embodiment, the portable memory device might involve a standard USB “thumb drive”. The thumb drive might be plugged into a receptacle on the base station 210 for several nights to record data. It might then be removed and plugged into a standard USB port on host computer 220 for data uploading.

In any of the above embodiments for the second data connection 208, the host computer 220 may serve as a way station for the sleep data. It may utilize an internet connection to forward this data to a hosted web server 230, where the data may be stored in remote data storage 236 and used by a web based application for the generation of personalized sleep coaching tips and tricks for the user.

In another embodiment for the second data connection 208, the processed sleep data on the base station 210 may bypass the host computer 220 altogether, and may be uploaded directly to the hosted web server 230 via a wired or wireless interne connection 238. For example, the base station 210 may be plugged physically into a router via an Ethernet cable, or it may communicate wirelessly with a WiFi router. Alternatively, the base station 210 may be equipped with a radio that utilizes a wide area network for data upload via a cellular protocol such as GPS/CPRS, EDGE, UNITS, HSDPA, CDMA, EVDO, WIMAX and the like.

Once the data is uploaded to the hosted web server 230, the data may be fed into a processor running a Sleep coaching Program (SCP) Algorithm 234, which may analyze the data and generate a first set of sleep parameter changes for improving user sleep satisfaction. In addition to the processed sleep data, the user may also use a user interface 212 or 222 to answer survey questions about their sleep habits, note specific habits or events surrounding their sleep, and to keep a sleep diary. The survey and optional sleep diary information provide information about physical and psychological characteristics of the user. The SCP algorithm 234 takes all this information into consideration to generate an interactive sleep coaching program or first set of advice for improving user sleep satisfaction in the form of a set of customized, step by step instructions 232, with the object of coaching the user to improve his or her sleep satisfaction over time. In certain embodiments, the user-provided sleep behavior and characteristics may be stored in a first computer memory database 236a in remote data storage 236. In certain embodiments, the first database may be located in local storage 216, 224, or at any other location with storage capabilities.

In certain embodiments, a second computer memory database 236b may store sleep-related data such as information relating sleep parameters or sleep parameter changes to quality of sleep. For example, second database 236b may contain information about optimal sleep requirements as a function of age, information relating consumed caffeine quantities to sleep parameters, information about the effects of increasing deep sleep time on total sleep quality, and other data relating sleep parameters, sleep parameter changes, or user behavior to sleep quality. In certain embodiments, a third computer memory database 236c may store sleep-related advice such as advice for improving user sleep satisfaction. For example, third database 236c may contain information and advice about reducing caffeine or alcohol consumption to improve sleep satisfaction, such as the amount of caffeine or alcohol consumption allowable before adverse effects are seen in sleep parameters or daytime subjective or objective parameters, or how long before bedtime caffeine or alcohol consumption should be stopped for improved sleep satisfaction. User sleep quality may be measured in terms of sleep-related parameters such as the ZQ factor, calculated as shown below. In certain embodiments, second database 236b and third database 236c may be located in remote data storage 236. In other embodiments, second database 236b and third database 236c may be located in local storage 216, 224, or any other location with storage capabilities. In certain embodiments, second database 236b and third database 236c are located in different storage areas.

In certain embodiments, the first, second and third databases may be combined into at least one database. This at least one database may be stored at the base station 210, the host computer 220, the web server 230, or any other location with storage capabilities.

In any of the above embodiments, the sleep coaching program algorithm may use data from the first, second, and third databases to generate the interactive sleep coaching program.

The Graphical User Interface (GUI) for the sleep coaching program algorithm 234 may be displayed via a web browser on user interface 222, where pertinent sleep data may be presented to the end user utilizing specific user interface constructs that make it easy for end users to understand sleep data.

In an exemplary embodiment, the user uses a secure login mechanism to access his or her personal sleep data hosted on the web server 230. All the data is centralized on the server 230 and backed up routinely. This implementation may allow the user to access his or her own data, as well as access a variety of community tools, such as a sleep forum, on line chat with a sleep coaching professional, and a variety of other features available over the internet.

In addition to physiological variables and lifestyle factors, environmental cues may also be tracked over the course of time as the environment may have an effect on a person's sleep. These factors may be tracked automatically using sensors (not shown in figure) within the sensor modules 202, 204, and/or 206, or base station 210, or by the user through a user interface (not shown in figure). Factors that may be tracked include, but are not limited to, light, sound, temperature, and humidity. These factors may be tracked over time and compared to a user's sleep over the course of a night or compared over many nights in order to track correlations with these factors and the user's sleep quantity and quality. It can also be integrated into the sleep coaching plan to provide advice.

In certain embodiments, the host computer 220 and the hosted web server 230 may be combined. This combination of host computer 220 and server 230 may be located either local to the user or at a central location geographically remote from the user. This central location may be geographically distant from any individual user but also be accessible to multiple users through, for example, an internet interface.

FIG. 3 depicts a system architecture block diagram 300 for an exemplary data driven sleep coaching system, according to an illustrative embodiment of the invention. In one embodiment, the sensor module 302 may comprise a sensor housing that houses a set of dry fabric electrodes (not shown). Signals from the sensors 304 may be passed through an analog filter and gain 306, then sent to a data acquisition module 308. The digitized signal may then pass to a microcontroller 310 on board the electronics module (not shown). There is a battery power source 322 and on board storage 312 for the electronics module to cache data during data capture. Software running within the microcontroller 310 breaks up the stream of incoming data into data packets, and sends it wirelessly to the base station 330 via a radio frequency transmitter 316 connected to an antenna 318. In certain embodiments, there may be a wired communication module 320 that allows the headband to communicate with the base station through headband wired communication module 344. There may also be a headband charging module 350 that allows the headband 302 to be charged by the base station 330.

In one embodiment, the first data transfer mechanism 324 may be implemented as a wireless connection. The packetized data may be sent wirelessly to the base station 330, which may be received via a radio frequency receiver 340 connected to an antenna 338. For example, an unregulated, 2.4 GHz frequency band may be used with a proprietary protocol for data transmission.

In one embodiment, these packets are sent to a microcontroller 342 on board the base station 330. The base station 330 may have user input elements 332 such as buttons, and a user display 334 such as an LCD display. In certain embodiments, base station 330 may have an audio device 336 to present sounds and alerts to the user. The base station 330 may also have on board storage 348 for caching sleep data, as well as a receptacle for a removal portable memory device such as an SD card for continuous data collection over several nights (not shown). The power source 352 of base station 330 may be based on batteries, either nonrechargeable or rechargeable, or based on power from a wall plug. The received packets of raw EEG data may be analyzed by software running on the microcontroller 342, such as a sensor data analysis software module (not shown). The sensor data analysis software module may break the EEG data up into frequency bands and then into sleep stages. Additional sleep data may calculated by the microcontroller 342 and stored in on-board storage 348.

In certain embodiments, the base station 330 may function as a standard alarm clock with a wake algorithm that is optionally keyed to an optimal wake theory. Sleep science indicates that the optimal time to wake a user from sleep is during REM or light sleep. Waking a user during deep sleep may result in excessive sleep inertia. The base station has access to sleep data collected throughout the night, and is therefore optionally able to sound an alarm during an optimal wakeup window given a user-specified latest wake time. An optional backup battery (not shown) may be used to guarantee that the alarm clock keeps its time even in the event of a power outage or a brownout event.

In certain embodiments, data connection 356 may comprise the physical transfer of a removable portable memory device (not shown). The removable portable memory device, such as an SD card, may be used to transfer nights of data to a host computer 360 connected to a data transfer means 372 such as a card reader. In certain embodiments, the sleep coaching program may be implemented as a hosted web based application, where the actual algorithm runs on a processor such as server computer 386 in remotely located server 380, and the output may be presented to the user on a web browser 376. The data may be uploaded to the remotely located server 380 over an interact connection 378. The data may be stored and backed up on data storage 384 located on the server 380. In certain embodiments, the first computer memory database 384a may store user behavior and characteristics data, the second computer memory database 384b may store sleep-related data, and the third computer memory database 384c may store sleep-related advice. In certain embodiments, one or more of these databases may also be located in base station 330, host computer 360, or elsewhere. A hosted, web based application 382 running on a processor such as server computer 386 in the server 380 may incorporate an implementation of the sleep coaching program algorithm. The algorithm may analyze the uploaded sleep data on a per user basis, and may generate a step by step sleep plan for the user. This plan may then be transmitted back to the host computer 360 via an interne connection 378, and presented to the user via a web browser 376, using standard peripherals such as a visual display 364, auditory output 366 and a user input 362 such as a computer keyboard and keys for user interaction.

In alternate embodiments, the sleep coaching algorithm may be implemented as a standalone desktop application that runs directly on a processor such as host processor 374 in the host computer 360. The application may present a graphical user interface to the user. Data storage and backup may be done locally on the host computer 360.

In another embodiment, the sensor module may directly transmit raw sensor data to a host computer via a data connection means. The sensor data analysis software module may be implemented either on the host computer as a desktop application, run by host processor 374, or implemented as a web application running on server computer 386. In the first example, where the data analysis software module is implemented as a desktop application, raw sensor data may be analyzed and processed into sleep data that is usable by the sleep coaching program algorithm and presented to the user as well. This reduces the amount of data that needs to be uploaded via the internet and may present a faster end user workflow. In the second example, where the data analysis software module is located on the server, the raw sensor data may be transmitted over the internet to the server. The advantage of this implementation is the consolidation of analysis software on one platform which may be updated and serviced on an as needed basis without involving user input.

In yet another embodiment, the microcontroller 310 in sensor module 302 may be augmented to include the sensor data analysis software module and provide a way to upload processed sleep data to the host computer 360 via a data transfer mechanism (not shown), again eliminating the base station 330.

### Sleep Metrics

In certain embodiments, sleep metrics may be calculated by the sensor data analysis module. These sleep metrics may be saved as the sleep data for the user. Various combinations of these sleep metrics may be presented to the user, either on the display 334 of the base station 330 or as part of the GUI displayed within a web browser 376 on the host computer 360. FIG. 4 depicts an illustrative representation of sleep metrics presented on a display, according to an illustrative embodiment of the invention. The sleep metric shown in FIG. 4 is a hypnogram (see below). [Hi—Then what is it?] The following are examples of some possible sleep metrics, and is not a comprehensive list.

Total Z

The total amount of sleep may be calculated with the following formula:

Total sleep time (Total Z)=Time in Bed (TiB)−Time in Wake (TiW)−Time to Sleep (Time to Z)

Time to Z

The time taken for the user to fall asleep may also be calculated as Time to Sleep (Time to Z)

Bed Time and Rise Time

The clock time when a user goes to bed and when a user gets up from bed may be calculated as Bed Time and Rise Time. In one embodiment, where a physiological signal is recorded during the night, the detection of the beginning of signal collection may be used to signify bed time, and the detection of the end of signal collection may be used to signify rise time. Signal collection start and end may be defined as whether the sensors are receiving a recognizable physiological signal from a user, as opposed to white noise from the environment.

Sleep Stage Breakdown

The actual time spent in each stage of sleep, as well as the percentage breakdown, may also be calculated. The stages of sleep include: Wake; Rapid Eye Movement (REM); Light (includes Stages 1 and 2) and Deep (includes Stages 3 and 4). Thus the time spent in each sleep stage may be calculated as follows. The same information for the time spent in each sleep stage may be presented as a percentage of total sleep time.

Time in Wake

Time in REM

Time in Light

Time in Deep

Number of Awakenings

The number of awakenings affects how a user feels when he or she gets up in the morning, and is also used as a sleep data metric.

Hypnogram

The sleep stage as a function of time for the duration of the night may be presented to the user in the form of a hypnogram, which is presented as a bar chart where the height of each bar depicts the stage of sleep. Each bar may represent a predetermined sampling duration (e.g. 5 minutes) during the night. An exemplary depiction of a hypnogram is shown in FIG. 4.

The ZQ

The overall sleep quality may be presented to the user as a single number, the ZQ, which takes into account both the duration of sleep, times awakened, and time spent in each stage of sleep. In an exemplary embodiment, the ZQ may be calculated with the following formula:

ZQ=8.5*(Total Z) +0.5*(Time in REM) +1.5*(Time in Deep) −0.5*(Time in Wake) 0.07*(Number of Awakenings)

Any combination of the above information may be presented on a night-by-night basis, or it can also be viewed over time by the user. For example, the user may be interested in looking at how the Total Z changes over the course of several weeks. Alternatively, the user might be interested in investigating how the breakdown of sleep stages for a night changes over time, to see if he or she is experiencing an increase in restorative sleep (REM and deep) as opposed to light sleep. The user can also be presented data not as a function of rather as it correlates with other data available. For example, if a user records in a journal data which shows caffeine usage that information can be presented as a function of caffeine usage and time to fall sleep.

This information may be presented in a variety of ways. FIG. 4 shows one example, where some of the information is presented on the display of the base station. In certain embodiments, the same information may be presented in a graphical user interface (GUI) on the host computer, whether as part of a desktop application or as a web browser based application.

In certain embodiments, some of the information may be presented on the base station (e.g. night by night data and simple trend data over several nights), while more data viewing and analysis options may be available on the host computer (e.g., detailed trend analysis of sleep stages, time to Z and the like). Additional trend information may be displayed as line charts, pie charts, tables and other graphical presentations on the host computer (not shown).

FIG. 5 depicts a high level overview of the sleep coaching program (SCP) according to an embodiment. The SCP is a program that helps users get a better night's sleep by leveraging the unique values offered by sleep data collection and analysis, coupled with an interactive online environment with a rich multimodal user interface. The basic tenets that dictate the SCP include:
Personalization/Customization—the user should feel that e SCP caters to them as an individual.
Simplicity—the interface should be intuitive, instructive, and informative, without overwhelming the user.
Education—the user should learn material that will help them continue to experience the benefits of the SCP even if they end their participation.
Scientific Integrity—the SCP should be grounded within a theoretical framework that can be supported by the scientific community, both in sleep and in behavior.
Effectiveness—the SCP should provide users with an educational experience that empowers them to improve their lives in an effort to improve their sleep satisfaction.

In one embodiment, the sleep coaching program (SCP) may be implemented as a step-wise program. In this type of approach, the user is guided through a number of steps to improve their sleep. Within each step, the user may be given educational and instructional materials as well as clear directions on what they should do to complete the tasks within each step. In certain embodiments, a predetermined target elapsed time (e.g. 14 days) may also be set, to help pace the user through the program and to ensure some level of closure over a given period of time.

The following example, depicted in FIG. 5, illustrates how this type of approach may be implemented as a 4-step program 500.

1. Profiling the User's Sleep (step 502)

The purpose of this step is to profile or categorize a user based on their lifestyle habits and sleep profile. A user should complete this process in order to get personalized feedback. The specific tasks involved in this step comprise entering pertinent information about their demographics (male or female, as well as age range) (step 504), answering questions about their lifestyle (step 506), and answering questions (step 508) that describe what type of sleeper they are or would like to be and what goals they have for sleep and lifestyle satisfaction. (step 510). In one exemplary embodiment, the user may be guided through answering key questions as part of the account sign up and/or login process. This approach has the benefit of providing the user with immediate positive reinforcement by completing the first step of the program simply by signing up for the program. This encourages the user to stay engaged in the program and improves the overall probability of success for the user.

2. Collect Sleep Data from a Single Night's Sleep (step 512)

In this step, the user is introduced to the equipment and data collection approach used in this program, which may comprise a sensor module such as a headband with adjustable straps for attaching electrodes to the forehead of the user to collect EEG data during their sleep and a base station for storing and analyzing the raw sensor data and a data connection to upload the data to a computer. The user may learn about the program and the equipment by browsing through multimedia tutorials, FAQs and other didactic materials. They are then tasked with actually going to bed while wearing the sensors and collecting the data for one night. In one embodiment, an SD card or other portable storage device may be inserted in the base station to store the sleep data for future uploading. Upon awakening, the user is encouraged to fill in a sleep diary where they record their consumption of various substances such as caffeine and alcohol, their activities (such as any rigorous exercise within two hours of bed time), and other factors that might affect the quality and quantity of their sleep.

3. Upload Data and Fill in a Sleep Diary (step 514)

In this step, the user may upload the data using a data transfer means, and interact with relevant parts of the web interface for the sleep coaching program to review their sleep data as well as receive personalized instructions for the sleep coaching program. In one embodiment, the user may extract the SD card or portable storage device from the base station and insert it into a card reader connected to a personal computer running a web based interface for the sleep coaching program. The user may be taken through the upload process via an interactive tutorial and completes their first data upload. The user may be prompted to fill in their sleep diary for the first time.

4. Sleep Workshops (step 516)

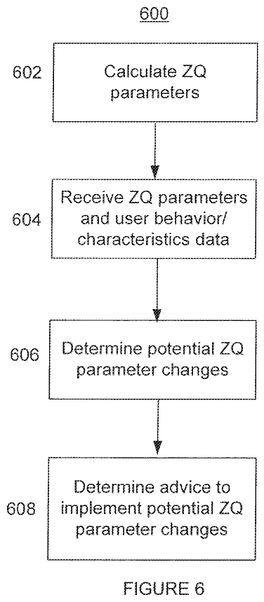

In this step, the concept of sleep workshops may be presented to the user. The sleep coaching program may use the data gathered in the previous steps to create a set of personalized advice that helps the user understand what factors affect the quality and quantity of their sleep, and what they can do to effect positive change. FIG. 6 depicts a flowchart 600 for the creation of a set of personalized advice for improving sleep satisfaction according to an embodiment. In this embodiment, the creation of the set of personalized advice for improving sleep satisfaction may be begin by calculating; the ZQ factor described above (step 602). Once the ZQ factor is calculated, the various parameters in the ZQ equation may be examined in light of collected user behavior and characteristics data (step 606) in order to determine parameter changes that may optimize the achievable ZQ factor (step 608). For example, if the ratio of Time in Wake to Total Z for a particular user is lower than a particular threshold, and the user behavior data includes a particular behavior that tends to increase the time a sleeper is awake, then the system may suggest that the user reduce the particular behavior (step 608). In certain embodiment, the user may be presented with a number of workshops, each of which is targeted to address a particular issue identified in the sleep habits and sleep data of the user. The user may choose which workshops he or she would like to follow (step 518). For each workshop, the user may start by responding to a questionnaire that provides more in-depth questions about the topic covered in that workshop (step 520). Then the user may be given a number of tips (e.g. four tips) (step 522). The user should try to follow some proportion of these tips (e.g. three out of four) over the course of a predetermined interval of time (e.g. at least three nights). Data may be collected throughout the workshop, and uploaded on an ongoing basis. At the end of the workshop, a summary of the steps taken and the results achieved may be presented to the user (step 524). Information may be presented in a multimedia fashion with text, video clips, images, audio clips, interactive quizzes and so on. The user may be prompted to collect data for a specified minimum duration of time in order to accumulate adequate baseline data to generate a customized sleep coaching program.

The user may then repeat the process for any other selected workshops where they work on a different aspect of their sleep. By the end of the workshop phase, the user should have proactively worked on trying to improve several factors that may affect their sleep, and may have data and sleep diary entries to indicate whether or not the steps taken resulted in better sleep satisfaction for the user. Once a user finishes all the steps in this program, they may continue to monitor their sleep and they may also re-engage in the stepwise program, returning to step 502, to reassess their current state of sleep, and to come up with new data that will craft a new customized sleep coaching program with workshops targeted at improving different factors that affect their sleep at the current time. In this way, the user employs a progressive process for collecting sleep data over a subsequent period of time and getting from the system a second set of sleep advice for improving the sleep satisfaction, where the new advice is based at least in part on the sleep data associated with the second later period of time and the first set of advice given to the user.

In certain embodiments, the sleep parameters and workshops may be generated automatically by the system. In certain embodiments, a sleep expert may also provide input in the generation of sleep parameters, workshops, or otherwise contact the user.

Note that the exact number of steps and the exact contents within each step is illustrative only. The overarching invention is that this is a program that takes a user through different types of tasks in a process to educate them about sleep, collect information about how they sleep, and develop strategies to help them improve their sleep satisfaction. Other specific implementations may involve a different number of steps, different separation for the contents between each step, or different content for each step altogether. Examples of other possible steps follow (not shown):

Try a Quality-of-Sleep Indicator—the ZQ Simulator

In this step, the user may be educated about specific sleep metrics used by the sleep coaching program to gauge the quality and quantity of sleep. The user may experience an interactive simulator, where they can change certain parameters such as duration of sleep, time to fall asleep, amount of caffeine consumed within 2 hours of sleep and other such examples, and see if and how each change affects their sleep. The metrics used to gauge sleep may include: total duration of sleep; time to fall asleep; times awakened; time spent awake during the night of sleep; and a single score summarizing the quality of sleep in an easy to understand, linear metric. The quality of sleep may be presented as a single index (e.g. called the ZQ in an example implementation).

Sleep Style.

This step may be an opportunity for the user to provide more information about their particular sleep style and attitudes about sleep. This section may be composed of interactive questionnaires or quizzes, for example, so that the user can input data about their beliefs about the way they sleep. This data may be compared to physiological data that has been collected or may be later used to help determine the workshops offered to the user or the bed/rise times that are calculated to optimize the user's sleep schedule.

Recommending Bed and Rise Time

Based on collected sleep information, a suggested optimal bed or rise time may be calculated and suggested to the user. The user may be advised to follow the bed/rise time recommendation every day, and to choose the bed and rise times such that they get an adequate amount of sleep during the night.

Final Report

This step may be the conclusion of the program. A summary of the user's participation in the sleep coaching program may be provided to the user. The user may enter into a maintenance mode, much like the approach taken by weight loss programs such as Weight Watchers®. Incentives may be provided to the user to continue to use the device and website to quantify their sleep quality and to prevent any regression in the progress made to address their sleep problems.

The invention may be embodied in other specific forms without departing from the spirit or essential characteristics thereof. The foregoing embodiments are therefore to be considered in all respects illustrative, rather than limiting of the invention, and various modifications can be made by those skilled in the art without departing from the scope and spirit of the invention.

1. A sleep coaching system, comprising:
at least one physiological sensor configured to be mounted to a portion of a user's head, the at least one physiological sensor configured to measure at least one physiological signal associated with the user, wherein the at least one physiological sensor comprises at least one electrically conductive bio-sensor for sensing electro-physiological signals of the user's head; and
a first processor configured to:
correlate data associated with the at least one physiological signal with input data that is indicative of one or more aspects of the user's behavior that affect the user's sleep; and
generate a first set of sleep-related advice from a database for improving the user's sleep satisfaction based on the data associated with the at least one physiological signal and the input data of the user.

2. The sleep coaching system of claim 1 further comprising a user interface configured to prompt for, and receive, the input data.

3. The sleep coaching system of claim 1, wherein the first processor is contained in a computing device located locally with respect to the at least one physiological sensor.

4. The sleep coaching system of claim 3, further comprising a user interface configured to prompt for, and receive, the input data, and wherein the user interface is incorporated into the computing device.

5. The sleep coaching system of claim 1, wherein the first processor is contained in a server located remotely with respect to the at least one physiological sensor.

6. The sleep coaching system of claim 1, further comprising a base station located locally with respect to the at least one physiological sensor, the base station configured to communicate with the at least one physiological sensor and the first processor and record data associated with the at least one physiological sensor.

7. The sleep coaching system of claim 6, wherein the base station is a table top unit.

8. The sleep coaching system of claim 6, wherein the base station includes a second processor configured to generate a baseline measure representative of sleep quality of the user's sleep based on the at least one physiological signal.

9. The sleep coaching system of claim 8, wherein the user interface includes a display configured to present at least one of the at least one physiological signal, the baseline measure, and/or the first set of sleep-related advice to the user.

10. The sleep coaching system of claim 8, wherein the recorded data is recorded over a first period of time and the first processor is configured to determine sleep data over a second later period of time and provide a user a second set of sleep-related advice for improving the user's sleep satisfaction based in part on the sleep data associated with the second later period of time and the first set of sleep-related advice.

11. The sleep coaching system of claim 10, wherein the first processor is configured to adjust the baseline measure as a function of the sleep data determined over the second later period of time to reflect changes in sleep.

12. The sleep coaching system of claim 1, wherein the first processor is configured to present a correlation of the data associated with the at least one physiological signal and the data received at the user interface.

13. The sleep coaching system of claim 1, wherein the at least one physiological sensor further comprises one or more physiological sensors for sensing one or more of movement, respiration, heart rate, heart rate variability, peripheral arterial tone, galvanic skin response, and/or temperature.

14. The sleep coaching system of claim 1, wherein the at least one physiological sensor is mounted to the portion of the user's head using one of a flexible or rigid headband.

15. The sleep coaching system of claim 1, wherein the at least one physiological sensor comprises an electrode configured to make contact with the user's skin when the at least one physiological sensor is mounted to the portion of the user's head.

16. The sleep coaching system of claim 1, wherein the user interface comprises a touchscreen.

17. The sleep coaching system of claim 1, further comprising a sensor data analysis module configured to process the at least one physiological signal to generate processed sensor data by using a Fast Fourier Transform (FFT) to convert the at least one physiological signal into constituent frequency bands of the at least one physiological signal, wherein the processed sensor data is provided to the first processor.

18. The sleep coaching system of claim 17, wherein the sensor data analysis module is configured to use a neural net approach to convert information associated with the constituent frequency bands into stages of sleep.

19. The sleep coaching system of claim 1, further comprising the database, wherein the first set of sleep-related advice is generated based on data from the database that contains sleep-related data and advice data.

20. The sleep coaching system of claim 1, wherein the first set of sleep-related advice is associated with one or more of bed time, rise time, and/or consumption of caffeine and/or alcohol.

21. A method of a sleep coaching system comprising:
measuring, by at least one physiological sensor configured to be mounted to a portion of a user's head, at least one physiological signal associated with the user's sleep, wherein the at least one physiological sensor comprises at least one electrically conductive bio-sensor for sensing electro-physiological signals of the user's head;
accessing input data that is indicative of one or more aspects of the user's behavior that affect the user's sleep; and
correlating, by a processor, data associated with the at least one physiological signal with the input data of the user to generate a first set of sleep-related advice for improving the user's sleep satisfaction based on the data associated with the at least one physiological signal and the input data of the user.
